(19) United States
(12) Patent Application Publication
(10) Pub. No.: US 2021/0108263 A1
(43) Pub. Date: Apr. 15, 2021

(54) Methods and Compositions for Preparing Sequencing Libraries

(71) Applicant: CELULA CHINA MED-TECHNOLOGY CO., LTD., Chengdu (CN)

(72) Inventors: Liang Yang, Chengdu (CN); Jun Feng, Chengdu (CN); Haichuan Zhang, San Diego, CA (US)

(73) Assignee: CELULA CHINA MED-TECHNOLOGY CO., LTD., Chengdu (CN)

(21) Appl. No.: 16/496,413

(22) PCT Filed: Mar. 20, 2017
(86) PCT No.: PCT/CN2017/077234

§ 371 Date: Sep. 20, 2019

(51) Int. Cl. C12Q 1/6874

(52) U.S. Cl. CPC C12Q 1/6874

(57) ABSTRACT

The present invention relates to methods and compositions for preparing sequencing libraries. The methods and compositions provided herein enables next generation sequencing library preparation using multiplex PCR with reduced primer dimer formation.

### FIELD OF THE INVENTION

The present invention relates to methods and compositions for preparing sequencing libraries. The methods and compositions provided herein enables next generation sequencing library preparation using multiplex PCR with reduced primer dimer formation.

### BACKGROUND OF THE INVENTION

Next generation sequencing (NGS) or massively parallel sequencing typically uses a library generated by multiplex-polymerase chain reaction (PCR). The process of preparation of sequencing libraries can significantly impact the quality and the output of sequencing data. Current methods for preparing DNA libraries for NGS are time consuming, prone to significant sample loss and primer dimer formation, and result in low coverage of the genetic material that is being sequenced.

Thus, there remains a need for better methods for preparing sequencing libraries. More specifically, there is a need for methods to reduce primer dimer formation in multiplex-PCR based library preparation.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention improves next generation sequencing workflows by providing highly multiplexed PCR with reduced primer dimer formation. The methods and compositions of the present invention reduce costs associate with NGS library preparation and the sample DNA utilization rate.

In some embodiments, the present invention provides a method of generating a next-generation sequencing library, the method comprising: a) providing a sample comprising nucleic acids, wherein at least some of said nucleic acids in said sample comprise target nucleic acid sequences; b) enriching said sample from step a) for said target nucleic acid sequences; c) performing a first multiplex PCR comprising target nucleic acid sequences to provide amplicons; d) enriching said sample from step c) for target amplicons; and e) performing a second multiplex PCR comprising said target amplicons, sequencing adaptors, and barcodes to form barcoded target amplicons, thereby generating a next-generation sequencing library.

In other embodiments, the present invention provides a method of generating a next-generation sequencing library, the method comprising: a) providing a sample comprising nucleic acids, wherein at least some of said nucleic acids in said sample comprise target nucleic acid sequences; b) enriching said sample from step a) for said target nucleic acid sequences; c) performing a first multiplex PCR comprising target nucleic acid sequences to provide amplicons; d) enriching said sample from step c) for target amplicons; e) performing a second multiplex PCR comprising said target amplicons, sequencing adaptors, and barcodes to form barcoded target amplicons, and f) enriching said barcoded target amplicons from step e), thereby generating a next-generation sequencing library.

In some embodiments, the target nucleic acid sequences comprise 1 to 300 nucleotides. In some embodiments, the enriching step comprises contacting the sample with magnetic beads, wherein said beads bind to target nucleic acid sequences in the sample; and separating the target nucleic acid sequences bound to said beads from the remaining sample. In other embodiments, the first or second multiplex PCR comprises more than one primer pair and a hot-start polymerase. In yet other embodiments, the primer pair comprises a universal sequence and a target sequence. In other embodiments, the amplicons comprise a universal sequence and a target sequence. In some embodiment, the enriching step comprises applying amplicons to a filter, wherein the filter substantially retains the amplicons but allows unconsumed primers and primer dimers to pass through the filter. In other embodiments, the filter is a PCR products filter. In yet other embodiments, the enriching step comprises applying amplicons, primer dimers and/or unconsumed primers to a filter to provide filtered amplicons, primer dimers and/or unconsumed primers and contacting said filtered amplicons, primer dimers and/or unconsumed primers with magnetic beads, wherein said beads bind to said filtered amplicons; and separating the filtered amplicons bound to said beads from primer dimers and/or unconsumed primers not bound to said beads.

In some embodiments, the second multiplex PCR comprises forward primers and reverse primers. In certain embodiments, the reverse primers comprise a sequencing adaptor and a universal sequence. In other embodiments, the reverse primers comprise a sequencing adaptor, a barcode sequence, and a universal sequence. In some embodiments, the forward primers comprise a sequencing adaptor and a universal sequence. In yet other embodiments, the forward primers comprise a sequencing adaptor, a barcode sequence, and a universal sequence. In some embodiments, the enriching said barcoded target amplicons comprises contacting the barcoded target amplicons, primer dimers and/or unconsumed primers with magnetic beads, wherein said beads bind to said barcoded target amplicons; and separating the barcoded target ampicons bound to said beads from primer dimers and unconsumed primers not bound to said beads.

In yet other embodiments, the enriching step comprises contacting the nucleic acids and target nucleic acids with magnetic beads, wherein said beads bind to said nucleic acids but do not bind to said target nucleic acids; and separating the nucleic acids bound to said beads from said target nucleic acids not bound to said beads. In other embodiments, the enriching step comprises contacting the target nucleic acids, primer dimers, dNTPs, and/or primers with a filter, wherein said filter retains target nucleic acids but not primer dimers, dNTPs, and/or primers. In yet other embodiments, the filter is a PCR products filter. In some embodiments, the enriching step comprises subjecting the target nucleic acids to gel electrophoresis, ethanol precipitation, or column chromatography. In other embodiments, the multiplex PCR comprises at least 100 target nucleic acid sequences, at least 500 target nucleic acid sequences, or at least 1,000 target nucleic acid sequences. In yet other embodiments, the first or second multiplex PCR is performed in less than 40 PCR cycles, less than 30 PCR cycles, less than 20 PCR cycles, or less than 15 PCR cycles. In some embodiments, the first or second multiplex PCR further comprises potassium phosphate. In other embodiments, the concentration of potassium phosphate in the multiplex PCR is at least 5 mM, at least 10 mM, or at least 15 mM. In still other embodiments, the concentration of primers in the multiplex PCR is at least 10 nM, at least 20 nM, or at least 40 nM.

In other embodiments, the methods of the present invention further comprise sequencing to detect a genetic variation. In some embodiments, the genetic variation is chromosomal aneuploidy. In other embodiments, the chromosomal aneuploidy is fetal chromosomal aneuploidy. In yet other embodiments, the target nucleic acids are from a fetus, a child, and/or an adult.

The present invention provides a sequencing library according to claim 1 for use in sequencing. In some embodiments, the sequencing is a second-generation sequencing or a third-generation sequencing. In other embodiments, the sequencing is selected from a group consisting of genomic DNA sequencing, target fragment trapping sequencing (e.g., exon trapping sequencing), single-strand DNA fragment sequencing, fossil DNA sequencing and sequencing of cell-free DNA in a biological sample. In still other embodiments, the biological sample is selected from the group consisting of blood, plasma, urine, or saliva.

These and other embodiments of the present invention will readily occur to those of ordinary skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 sets forth data showing size and quantity of library PCR products. The figure illustrates the removal of unconsumed primers and primer dimers following multiplex PCR using filters and magnetic beads of the present invention.

FIGS. 2A-B set forth data showing over-amplification of multiplex PCR leads to under-quantification of NGS library.

FIG. 3 shows the effects of potassium phosphate concentration on target DNA amplification during PCR.

FIG. 4 shows the effects of PCR primer concentration on target DNA fragment ratio.

FIG. 5 shows enrichment of short DNA targets using methods of the present invention.

FIG. 6 shows read length histograms of primer-dimer and target DNA sequencing data for various PCR polymerases.

FIGS. 7A-B show size and quantity of library PCR products. FIG. 7A shows size and quantity of library PCR products prepared using magnetic beads of the present invention. FIG. 7B shows size and quantity of library PCR products prepared using both filters and magnetic beads of the present invention.

### DESCRIPTION OF THE INVENTION

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting.

The use of “including,” “comprising,” or “having,” “containing,” “involving,” and variations thereof herein, is meant to encompass the items listed thereafter and equivalents thereof as well as additional items.

It must be noted that as used herein and in the appended claims, the singular forms “a,” “an,” and “the” include plural references unless context clearly dictates otherwise. Thus, for example, a reference to “a nucleic acid” includes a plurality of such nucleic acids, and to equivalents thereof known to those skilled in the art, and so forth.

The term “about,” particularly in reference to a given quantity, is meant to encompass deviations of plus or minus five percent.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. A cell may include a fixed cell or a live cell.

The terms “nucleic acid,” “nucleic acid molecule,” “polynucleotide,” and “oligonucleotide” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. There is no intended distinction in length between the terms “nucleic acid,” “nucleic acid molecule,” “polynucleotide,” and “oligonucleotide” and these terms will be used interchangeably.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and are extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and anti-sense strands).

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

The term “amplicon” refers to the amplified nucleic acid product of a PCR reaction or other nucleic acid amplification process (e.g., ligase chain reaction (LGR), nucleic acid sequence based amplification (NASBA), transcription-mediated amplification (TMA), Q-beta amplification, strand displacement amplification, or target mediated amplification). DNA amplicons may be generated from RNA by RT-PCR.

As used herein, the term “probe” or “oligonucleotide probe” refers to a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally. The “oligonucleotide probe” may contain at least one fluorescer and at least one quencher. Quenching of fluorophore fluorescence may be eliminated by exonuclease cleavage of the fluorophore from the oligonucleotide (e.g., TaqMan assay) or by hybridization of the oligonucleotide probe to the nucleic acid target sequence (e.g., molecular beacons). Additionally, the oligonucleotide probe will typically be derived from a sequence that lies between the sense and the antisense primers when used for nucleic acid amplification.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under conditions, generally where there is about 90% or greater homology.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The “melting temperature” or “Tₘ” of double-stranded DNA is defined as the temperature at which half of the helical structure of the DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41(GC)%(Marmur et al. (1962) J. Mol. Biol. 5:109-118).

As used herein, a “biological sample” refers to a sample of cells, tissue, or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, cells, muscles, joints, organs, biopsies and also samples of in vitro cell culture constituents including but not limited to conditioned media resulting from the growth of cells and tissues in culture medium, e.g., recombinant cells, artificial cells, and cell components.

The term “subject” includes any invertebrate or vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like, insects, nematodes, fish, amphibians, and reptiles. The term does not denote a particular age. Thus, both adult and newborn individuals are intended to be covered.

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of computer science, statistics, chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; Handbook of Experimental Immunology, Vols. I-IV (D. M. Weir and C.C. Blackwell, eds., 1986, Blackwell Scientific Publications); Maniatis, Tₘ et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th}edition, John Wiley & Sons; Ream et al., eds. (1998) Molecular Biology Techniques: An Intensive Laboratory Course, Academic Press); M. R. Green and J. Sambrook, et al. (2012) Molecular Cloning: A Laboratory Manual, 4^{th} edition, Cold Spring Harbor Laboratory Press; Newton & Graham, eds. (1997) PCR (Introduction to Biotechniques Series), 2^{nd} edition, Springer Verlag; J. Xu, ed. (2014) Next-generation Sequencing: Current Technologies and Applications, Caister Academic Press; Y. M. Kwon and S. C. Ricke, eds. (2011) High-Throughput Next Generation Sequencing: Methods and Applications (Methods in Molecular Biology), Humana Press; L. C. Wong, ed. (2013) Next Generation Sequencing: Translation to Clinical Diagnostics, Springer.

The present invention relates to the development of methods and compositions for preparing sequencing libraries. The methods and compositions provided herein enables next generation sequencing library preparation using multiplex PCR with reduced primer dimer formation (see Examples). The methods of preparing sequencing libraries provided by the present invention reduce sequencing costs, improve sample DNA utilization rate, and save time. The sequencing libraries produced using the methods and compositions of the present invention may be used to detect genetic conditions in biological samples, for example, fetal trisomy in maternal plasma.

### Samples/Nucleic Acids

The methods of the invention may be used to generate sequencing libraries by multiplex amplification (e.g., multiplex PCR) of nucleic acids. In some embodiments, nucleic acids (e.g., DNA or RNA) are isolated from a biological sample containing a variety of other components, such as proteins, lipids, and other (e.g., non-target) nucleic acids. Nucleic acid molecules can be obtained from any material (e.g., cellular material (live or dead), extracellular material, viral material, environmental samples (e.g. meta genomic samples), synthetic material (e.g., amplicons such as provided by PCR or other amplification technologies)), obtained from an animal, plant, bacterium, archaeon, fungus, or any other organism. Biological samples for use in the present invention include viral particles or preparations thereof. In some embodiments, a nucleic acid is isolated from a sample for use as a template in an amplification reaction (e.g., to prepare an amplicon library or fragment library for sequencing). In some embodiments, a nucleic acid is isolated from a sample for use in preparing a library of amplicons.

Nucleic acid molecules can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, maternal blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a source for nucleic acid for use in the technology, including forensic specimens, archived specimens, preserved specimens, and/or specimens stored for long periods of time. e.g., fresh-frozen, methanol/acetic acid fixed, or formalin-fixed paraffin embedded (FFPE) specimens and samples. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Nucleic acid molecules can be obtained, e.g., by extraction from a biological sample, e.g., by a vanity of techniques such as those described by Maniatis, et al (1982) Molecular Cloning: A Laboratory Manual, Cold Spring Harbor. N.Y. (see, e.g., pp. 280-281).

In some embodiments, the technology provides for the size selection of nucleic acids, e.g., to remove very short fragments or very long fragments. In various embodiments, the size is 1, 2, 3, 4, 5, 6, 7, 8, 9, 10, 20, 30, 40, 50, 100, 200, 300, 40, 500, 600, 700, 800, 1,000, 5.000, 10,000 bp or longer. In some embodiments, the size selection methods of the present invention may be used for positive of negative selection of nucleic acids. In some embodiments, negative selection is used to remove non-target nucleic acids from an admixture of target and non-target nucleic acids. In other embodiments, positive selection is used to capture and isolate target nucleic acids from an admixture of target and non-target nucleic acids.

In various embodiments, a nucleic acid is amplified. Any amplification method known in the art may be used. Examples of amplification techniques that can be used include, but are not limited to, PCR, multiplex PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), hot start PCR, nested PCR, in situ polony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid based sequence amplification (NABSA) Other amplification methods that can be used herein include those described in U.S. Pat. Nos. 5,242,794; 5,494,810; 4,988,617; and 6,582,938.

In some embodiments, amplification is performed to generate amplicons using MyTaq DNA polymerase from Bioline. In some embodiments, end repair is performed to generate blunt end 5′ phosphorylated nucleic acid ends using commercial kits, such as those available from Epicentre Biotechnologies (Madison, Wis.).

In some embodiments, the methods of the present invention may be uses for normalizing an amplicon panel, e.g., an amplicon panel library. An amplicon panel is a collection of amplicons that are related. e.g., to a disease (e.g., a polygenic disease), disease progression, developmental defect, constitutional disease (e.g., a state having an etiology that depends on genetic factors, e.g., a heritable (non-neoplastic) abnormality or disease), metabolic pathway, pharmacogenomic characterization, trait, organism (e.g., for species identification), group of organisms, geographic location, organ tissue, sample, environment (e.g., for metagenomic and/or ribosomal RNA (e.g., ribosomal small subunit (SSU), ribosomal large subunit (LSU), 5S, 16S, 18S, 23S, 28S, internal transcribed sequence (ITS) rRNA) studies), gene, chromosome, etc. For example, a cancer panel comprises specific genes or mutations in genes that have established relevancy to a particular cancer phenotype (e.g., one or more of ABL1. AKT1, AKT2. ATM, PDGFRA, EGFR, FGFR (e.g., FGFR1, FGFR2. FGFR3). BRAF (e.g., comprising a mutation at V600, e.g., a V600E mutation), RUNX1, TET2, CBL, EGFR, FLT3, JAK2, JAK3, KIT, RAS (e.g., KRAS (e.g., comprising a mutation at G12, G13, or A146, e.g., a G12A, G12S, G12C. G12D, G13D, or A 146T mutation), HRAS (e.g., comprising a mutation at G12, e.g., a G12V mutation). NRAS (e.g., comprising a mutation at Q61. e.g., a Q61R or Q61K mutation)), MET, PIK3CA (e.g., comprising a mutation at H1047. e.g., a H1047L. H1047L, or H1047R mutation). PTEN, TP53 (e.g., comprising a mutation at R248, Y126, G245, or A159. e.g., a R248W. G245S, or A 159D mutation), VEGFA, BRCA, RET, PTPN11, HNHF1A, RB1, CDH1, ERBB2, ERBB4, SMAD4, SKT11 (e.g. comprising a mutation at Q37), ALK, IDH1, IDH2, SRC, GNAS, SMARCB1, VHL, MLH1, CTNNB1, KDR, FBXW7. APC, CSF1R, NPM1, MPL, SMO, CDKN2A, NOTCH 1, CDK4, CEBPA, CREBBP, DNMT3A, FES, FOXL2, GATA1, GNA11, GNAQ, HIF1A, IKBKB, MEN1, NF2, PAX5. PIK3R1, PTCH1, STK11, etc.). Some amplicon panels are directed toward particular “cancer hotspots”, that is, regions of the genome containing known mutations that correlate with cancer progression and therapeutic resistance.

In some embodiments, an amplicon panel for a single gene includes amplicons for the exons of the gene (e.g., 1, 2, 3, 4, 5, 6, 7, 8, 9, 10, 11, 12, 13, 14, 15, 16, 17, 18, 19, 20, or more exons). In some embodiments, an amplicon panel for species (or strain, sub-species, type, sub-type, genus, or other taxonomic level and/or operational taxonomic unit (OTU) based on a measure of phylogenetic distance) identification may include amplicons corresponding to a suite of genes or loci that collectively provide a specific identification of one or more species (or strain, sub-species, type, sub-type, genus, or other taxonomic level) relative to other species (or strain, sub-species, type, sub-type, genus, or other taxonomic level) (e.g., for bacteria (e.g., MRSA), viruses (e.g., HIV, HCV, HBV, respiratory viruses, etc.)) or that are used to determine drug resistance(s) and/or sensitivity/ies (e.g., for bacteria (e.g., MRSA), viruses (e.g., HIV, HCV, HBV, respiratory viruses, etc.)).

The amplicons of the panel typically comprise 50 to 1000 base pairs, e.g., in some embodiments the amplicons of the panel comprise approximately 50, 75, 100, 125, 150, 175, 200, 225, 250, 275, 300, 325, 350, 375, 400, 325, 350, 375, 400, 425, 450, 475, 500, 525, 550, 575, 600, 625, 650, 675, 700, 725, 750, 775, 800, 825, 850, 875, 900, 925, 950, 975, or 1000 base pairs. In some embodiments, an amplicon panel comprises a collection of amplicons that span a genome. e.g., to provide a genome sequence.

The amplicon panel is often produced through use of amplification oligonucleotides (e.g., to produce the amplicon panel from the sample) and/or oligonucleotide probes for sequencing disease-related genes. e.g. to assess the presence of particular mutations and/or alleles in the genome. In some embodiments, 10, 20, 30, 40, 50, 60, 70, 80, 90, 100, 150, 200, 300, 400, 500, 1000, or more genes, loci, regions, etc. are targeted to produce, e.g., 10, 20, 30, 40, 50, 60, 70, 80, 90, 100, 150, 20, 300, 400, 500, 1000, or more amplicons. In some embodiments, the amplicons are produced in a highly multiplexed, single tube amplification reaction (e.g., more than 1,000-plex PCR).

In some preferred embodiments, a number of amplification (e.g., thermal) cycles is minimized (e.g., m some embodiments, less than the number of cycles used in conventional technologies) to retain uniform coverage of target sequences by the amplicons, to provide accurate representation of target sequences in the amplicons, and/or to minimize and/or eliminate bias such as the bias introduced into amplified samples during the middle and late stages of amplification. In some embodiments, the number of amplification cycles is less than 40 cycles, less than 30 cycles, less than 20 cycles, or less than 15 cycles.

Nucleic acids to be amplified and sequenced may be genomic DNA or cDNA (i.e., derived from RNA by reverse transcription). Cell-free DNA or RNA may be amplified and used to generate sequencing libraries according to the methods of the present invention. Sources of nucleic acid molecules include, but are not limited to, organelles, cells, tissues, organs, and organisms. For example, a biological sample containing nucleic acids to be analyzed can be any sample of cells, tissue, or fluid isolated from a prokaryotic, archaeon, or eukaryotic organism, including but not limited to, for example, blood, saliva, cells from buccal swabbing, fecal matter, urine, bone marrow, bile, spinal fluid, lymph fluid, sputum, ascites, bronchial lavage fluid, synovial fluid, samples of the skin, external secretions of the skin, respiratory, intestinal, and genitourinary tracts, tears, saliva, milk, organs, biopsies, and also samples of cells, including cells from bacteria, archaea, fungi, protists, plants, and animals as well as in vitro cell culture constituents, including recombinant cells and tissues grown in culture medium. A biological sample may also contain nucleic acids from viruses. In certain embodiments, nucleic acids (e.g., DNA or RNA) are obtained from a single cell or a selected population of cells of interest, the cell may be a live cell or a fixed cell. In certain embodiments, the cell is an invertebrate cell, vertebrate cell, yeast cell, mammalian cell, rodent cell, primate cell, or human cell. Additionally, the cell may be a genetically aberrant cell, rare blood cell, or cancerous cell, the target nucleic acids may be from a fetus, a child, or an adult.

### Enriching Methods

The methods and compositions of the present invention may be used to enrich target nucleic acids or amplicons for sequencing libraries. Enrichment methods utilized in the present invention may include use of magnetic beads of filters.

In some embodiments, target nucleic acids or amplicons are enriched using PCR filters. Such PCR filters include PCR plates that use a size-exclusion membrane and vacuum filtration. The method typically comprises loading a sample comprising nucleic acids and/or amplicons into a well containing a size-exclusion membrane, filtering the sample in the well with a vacuum, and then adding a buffer to the well to recover the nucleic acids and/or amplicons. In some embodiments, the sample comprises primer dimers and/or unconsumed primers that will pass through the filer membrane and be separated from target nucleic acids and/or amplicons

### Buffers and Reagents

In the methods of the present invention, the mixture comprising nucleic acids (e.g., amplicons) and magnetic beads is maintained under conditions appropriate for binding of the nucleic acids to the functional groups on the beads. In some embodiments, the methods and agents (reagents) described herein are used together with a variety of purification techniques (e.g., nucleic acid purification techniques) that involve binding of nucleic acid to beads (e.g., solid phase carriers), including those described in. e.g., U.S. Pat. Nos. 5,705.628; 5,898,071; 6,534,262; WO 99/58664; U S. Pat. Appl. Pub. No. 2002/0094519 A 1. U.S. Pat. Nos 5.047.513; 6.623.655; and 5,284,933, the contents of which are herein incorporated by reference.

As described herein, one or more agents (e.g., buffers, enzymes) is/are used to bind or remove the nucleic acids (e.g., amplicons) from the magnetic beads. In various embodiments, the components of the agents that promote association (e.g., binding) and/or disassociation of the target nucleic acids with the magnetic beads are present in one agent or in multiple agents (e.g., a first agent, a second agent, a third agent, etc.) Accordingly, when more than one agent is used in the methods of the present invention, embodiments provide that the agents are used simultaneously or sequentially. Depending on the purpose for which the methods described herein are used, one of skill m the art can determine the number and order of agents to be used in the methods of the present invention.

In some embodiments, the agent is used in the methods of the present invention to cause the nucleic acids (e.g., amplicons) in the mixture to precipitate or adsorb onto the functional groups on the surface of the magnetic beads (a nucleic acid precipitating agent). In one embodiment, a nucleic acid precipitating agent is used at a sufficient concentration to precipitate the nucleic acid of the mixture onto the magnetic beads.

A “nucleic acid precipitating reagent” or “nucleic acid precipitating agent” is a composition that causes a nucleic acid to go out of solution. Suitable precipitating agents include alcohols (e.g., short chain alcohols, such as ethanol or isopropanol) and poly-OH compounds (e.g., a polyalkylene glycol). The nucleic acid precipitating reagent can comprise one or more of these agents. The nucleic acid precipitating reagent is present in sufficient concentration to bind the nucleic acid onto the magnetic beads nonspecifically and reversibly. Such nucleic acid precipitating agents can be used, for example, to bind nucleic acids non-specifically, or nucleic acids specifically, depending on the concentrations used, to magnetic beads, e.g., magnetic beads comprising COOH as a functional group.

In one embodiment, carboxy-based magnetic beads are used that involve binding nucleic acids to carboxyl coated solid phase carriers (e.g., magnetic and/or paramagnetic microparticles) using various nucleic acid precipitating reagents or crowding reagents such as alcohols, glycols (e.g., alkylene, polyalkylene glycol, ethylene, polyethylene glycol), and polyvinyl pyrrolidinone (PVP) (e.g., polyvinyl pyrrolidinone-40). In some embodiments, the molecular weights of these precipitating and/or crowding reagents are adjusted to produce low viscosity solutions with substantial precipitating power. In some embodiments, size-specific nucleic acid isolation is performed by either adjusting the concentration of the precipitating and/or crowding reagents, the molecular weight of the precipitating and/or crowding reagents, or by adjusting the salt, pH, polarity, or hydrophobicity of the solution. Large nucleic acid molecules are precipitated and/or crowded out of solution at low concentrations of salt, precipitating, and/or crowding reagents, whereas the smaller nucleic acid molecules are precipitated and/or adsorbed at higher concentrations of precipitating and/or crowding reagents. See, for example, U.S. Pat. Nos. 5,705,628; 5,898,071; 6,534,262 and U.S. Published Application No 2002/0106686, all of which are incorporated herein by reference.

Appropriate alcohol (e.g., ethanol, isopropanol) concentrations (final concentrations) for use in the methods of the present invention are from approximately 5% to approximately 100%; from approximately 40% to approximately 60%; from approximately 45% to approximately 55%; and from approximately 50% to approximately 54%, described as a volume:volume ratio.

Appropriate polyalkylene glycols include polyethylene glycol (PEG) and polypropylene glycol. Suitable PEG can be obtained from Sigma (Sigma Chemical Co., St. Louis Mo., Molecular weight 8000, Dnase and Rnase free, Catalog number 25322-68-3). The molecular weight of the polyethylene glycol (PEG) can range from approximately 250 to approximately 10.000, from approximately 1000 to approximately 10.000; from approximately 2500 to approximately 10.000; from approximately 6000 to approximately 10.000; from approximately 600 to approximately 8000; from approximately 7000 to approximately 9000; from approximately 8000 to approximately 10,000. In general, the presence of PEG provides a hydrophobic solution that forces hydrophilic nucleic acid molecules out of solution. In one embodiment, the PEG concentration is from approximately 5% to approximately 20%. In other embodiments, the PEG concentration ranges from approximately 7% to approximately 18%; from approximately 9% to approximately 16%; and from approximately 10% to approximately 15%, described as a weight:volume ratio.

Optionally, salt may be added to the reagent to cause precipitation of the nucleic acid in the mixture onto the magnetic beads. Suitable salts that are useful for facilitating the adsorption of nucleic acid molecules targeted for isolation to the magnetically responsive microparticles include sodium chloride (NaCl), lithium chloride (LiCl), barium chloride (BaCl₂), potassium chloride (KCl), calcium chloride (CaCl₂), magnesium chloride (MgCl₂), and cesium chloride (CsCl). In some embodiments, sodium chloride is used. In general, the salt minimizes the negative charge repulsion of the nucleic acid molecules. The wide range of salts suitable for use in the method indicates that many other salts can also be used and suitable levels can be empirically determined by one of ordinary skill in the art. The salt concentration can be from approximately 0.005 M to approximately 5 M, from approximately 0.1 M to approximately 0.5 M; from approximately 0.15 M to approximately 0.4 M; and from approximately 2 M to approximately 4 M.

In embodiments in which the functional group is a sequence that is complementary, and thus hybridizes, to one or more nucleic acids in the mixture, a hybridizing buffer can be used for binding. Suitable buffers for use in such a method are known to those of skill in the art. An example of a suitable buffer is a buffer comprising NaCl (e.g., approximately 0.1 M to approximately 0.5 M), Tris-HCl (e.g., 10 mM), EDTA (e.g., 0.5 mM), sodium citrate (SSC), and combinations thereof.

A suitable “elution buffer” for use in the methods of the present invention is a buffer that elutes (e.g., selectively) target nucleic acid from the functional group(s) of the magnetic beads. In some embodiments, the elution buffer is water or an aqueous solution. For example, useful buffers include, but are not limited to, Tris-HCl (e.g., 10 mM, pH 7.5), Tris acetate, sucrose (20% w/v), EDTA, and formamide (e.g., at 90% to 100%) solutions. In some embodiments, the elution buffer is a buffered salt solution comprising a monovalent (one or more) cation such as sodium, lithium, potassium, and/or ammonium (e.g., from approximately 0.1 M to approximately 0.5 M). Elution of nucleic acid from the solid phase carrier can occur quickly (e.g., in thirty seconds or less) when a suitable low ionic strength elution buffer is used.

In addition, impurities (e.g., proteins (e.g., enzymes), metabolites, chemicals, unincorporated nucleotides and/or primers, or cellular debris) can be removed from the magnetic beads by washing the magnetic beads with nucleic acid bound thereto (e.g., by contacting the magnetic beads with a suitable wash buffer solution) before separating the magnetic bead-bound target species from the magnetic beads. As used herein, a “wash buffer” is a composition that dissolves or removes impurities that may be bound to a microparticle, associated with the adsorbed nucleic acid, or present in the bulk solution, but that does not solubilize the target nucleic acids absorbed onto the magnetic bead. The pH, solute composition, and concentration of the wash buffer can be varied according to the types of impurities that are expected to be present. For example, ethanol (e.g., 70% (v/v)) exemplifies a preferred wash buffer useful to remove excess PEG and salt. In one embodiment, the wash buffer comprises NaCl (e.g., 0.1 M), Tris (e.g., 10 mM), and EDTA (e.g., 0.5 mM). The magnetic beads with bound nucleic acid can also be washed with more than one wash buffer solution. The magnetic beads can be washed as often as required (e.g., one, two, three or more. e.g., three to five times) to remove the desired impurities. However, the number of washings is preferably limited to minimize loss of yield of the bound target species.

A suitable wash buffer solution has several characteristics First, the wash buffer solution must have a sufficiently high salt concentration (a sufficiently high ionic strength) that the nucleic acid bound to the magnetic beads does not elute from the magnetic beads, but remains bound to the microparticles. A suitable salt concentration is greater than approximately 0.1 M and is preferably approximately 0.5 M. Second, the buffer solution is chosen so that impurities that are bound to the nucleic acid or microparticles are dissolved. The pH, solute composition, and concentration of the buffer solution can be varied according to the types of impurities that are expected to be present. Suitable wash solutions include the following: 0.5×saline-sodium citrate (SSC; A 20×stock solution comprises 3 M sodium chloride and 300 mM trisodium citrate (adjusted to pH 7.0 with HCl)); 10 mM ammonium sulfate, 400 mM Tris pH 9, 25 mM MgCl₂ and 1% bovine serum albumin (BSA); 1-4 M guanidine hydrochloride (e.g., 1 M guanidine HC with 40% isopropanol and 1% Triton X-100); and 0.5 M NaCl. In one embodiment, the wash buffer solution comprises 25 mM Tris acetate (pH 7.8), 100 mM potassium acetate (KOAc), 10 mM magnesium acetate (Mg₂OAc), and 1 mM dithiothreitol (DTT: Cleland's Reagent). In another embodiment, the wash solution comprises 2% SDS, 10% Tween. and/or 10% Triton.

The components of the agents used in the methods of the present invention can be contained in a single agent (reagent) or as separate components. In embodiments in which separate components of the agent(s) are used, the components may be combined simultaneously or sequentially with the mixture Depending on the particular embodiment, the order in which the elements of the combination are combined may not necessarily be critical. The nature and quantity of the components contained in the reagent are as described in the methods above. The reagent may be formulated in a concentrated form, such that dilution is desirable to obtain the functions and/or concentrations described in the methods herein.

Cells may be pre-treated in any number of ways prior to amplification and sequencing of nucleic acids (e.g., DNA and/or RNA). For instance, in certain embodiments, the cell may be treated to disrupt (or lyse) the cell membrane, for example, by treating samples with one or more detergents (e.g., Triton-X-100, Tween 20, Igepal CA-630, NP-40, Brij 35, and sodium dodecyl sulfate) and/or denaturing agents (e.g., guanidinium agents). In cell types with cell walls, such as yeast and plants, initial removal of the cell wall may be necessary to facilitate cell lysis. Cell walls can be removed, for example, using enzymes, such as cellulases, chitinases, or bacteriolytic enzymes, such as lysozyme (destroys peptidoglycans), mannase, and glycanase. As will be clear to one of skill in the art, the selection of a particular enzyme for cell wall removal will depend on the cell type under study.

After lysing, nucleic acid extraction from cells may be performed using conventional techniques, such as phenol-chloroform extraction, precipitation with alcohol, or non-specific binding to a solid phase (e.g., silica). Care should be taken to avoid shearing the nucleic acids to be sequenced during extraction steps. Additionally, enzymatic or chemical methods may be used to remove contaminating cellular components (e.g., ribosomal RNA, mitochondrial RNA, protein, or other macromolecules). For example, proteases can be used to remove contaminating proteins. A nuclease inhibitor may be used to prevent degradation of nucleic acids.

### PCR Methods

DNA may be amplified prior to sequencing using any suitable polymerase chain reaction (PCR) technique known in the art. In PCR, a pair of primers is employed in excess to hybridize to the complementary strands of a target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, N Y 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their Y ends face each other, each primer extending toward the other. Typically, the primer oligonucleotides are in the range of between 10-100 nucleotides in length, such as 15-60, 20-40 and so on, more typically in the range of between 20-40 nucleotides long, and any length between the stated ranges.

The DNA is extracted and denatured, preferably by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler (available from, e.g., Bio-Rad, Applied Biosystems, and Qiagen).

RNA may be amplified by reverse transcribing RNA into cDNA with a reverse transcriptase and then performing PCR (i.e., RT-PCR), as described above. Suitable reverse transcriptases include avian myeloblastosis virus (AMV) reverse transcriptase and Moloney murine leukemia virus (MMLV) reverse transcriptase (available from, e.g., Promega, New England Biolabs, and Thermo Fisher Scientific Inc.). Alternatively, a single enzyme may be used for both steps as described in U.S. Pat. No. 5,322,770, incorporated herein by reference in its entirety. In this manner, cDNA can be generated from all types of RNA, including mRNA, non-coding RNA, microRNA, siRNA, and viral RNA to allow sequencing of RNA transcripts.

In certain embodiments, amplification comprises performing a clonal amplification method, such as, but not limited to bridge amplification, emulsion PCR (ePCR), or rolling circle amplification. In particular, clonal amplification methods such as, but not limited to bridge amplification, emulsion PCR (ePCR), or rolling circle amplification may be used to cluster amplified nucleic acids in a discrete area (see, e.g., U.S. Pat. Nos. 7,790,418; 5,641,658; 7,264,934; 7,323,305; 8,293,502; 6,287,824; and International Application WO 1998/044151 A1; Lizardi et al. (1998) Nature Genetics 19: 225-232; Leamon et al. (2003) Electrophoresis 24: 3769-3777; Dressman et al. (2003) Proc. Natl. Acad. Sci. USA 100: 8817-8822; Tawfik et al. (1998) Nature Biotechnol. 16: 652-656; Nakano et al. (2003) J. Biotechnol. 102: 117-124; herein incorporated by reference). For this purpose, adapter sequences (e.g., adapters with sequences complementary to universal amplification primers or bridge PCR amplification primers) suitable for high-throughput amplification may be added to DNA or cDNA fragments at the 5′ and 3′ends. For example, bridge PCR primers, attached to a solid support, can be used to capture DNA templates comprising adapter sequences complementary to the bridge PCR primers, the DNA templates can then be amplified, wherein the amplified products of each DNA template cluster in a discrete area on the solid support.

In particular, the methods of the invention are applicable to digital PCR methods. For digital PCR, a sample containing nucleic acids is separated into a large number of partitions before performing PCR. Partitioning can be achieved in a variety of ways known in the art, for example, by use of micro well plates, capillaries, emulsions, arrays of miniaturized chambers or nucleic acid binding surfaces. Separation of the sample may involve distributing any suitable portion including up to the entire sample among the partitions. Each partition includes a fluid volume that is isolated from the fluid volumes of other partitions, the partitions may be isolated from one another by a fluid phase, such as a continuous phase of an emulsion, by a solid phase, such as at least one wall of a container, or a combination thereof. In certain embodiments, the partitions may comprise droplets disposed in a continuous phase, such that the droplets and the continuous phase collectively form an emulsion.

The partitions may be formed by any suitable procedure, in any suitable manner, and with any suitable properties. For example, the partitions may be formed with a fluid dispenser, such as a pipette, with a droplet generator, by agitation of the sample (e.g., shaking, stirring, sonication, etc.), and the like. Accordingly, the partitions may be formed serially, in parallel, or in batch, the partitions may have any suitable volume or volumes, the partitions may be of substantially uniform volume or may have different volumes. Exemplary partitions having substantially the same volume are monodisperse droplets. Exemplary volumes for the partitions include an average volume of less than about 100, 10 or 1_L, less than about 100, 10, or 1 nL, or less than about 100, 10, or 1 pL, among others.

After separation of the sample, PCR is carried out in the partitions, the partitions, when formed, may be competent for performance of one or more reactions in the partitions. Alternatively, one or more reagents may be added to the partitions after they are formed to render them competent for reaction, the reagents may be added by any suitable mechanism, such as a fluid dispenser, fusion of droplets, or the like.

In some embodiments of the present invention, the first or second multiplex PCR includes the use of potassium phosphate. In certain embodiments, the concentration of potassium phosphate in the multiplex PCR is at least 5 mM, at least 10 mM, or at least 15 mM, the inventors have demonstrated that use of potassium phosphate in the methods of the present invention improves coverage of target DNA amplification during multiplex PCR.

In some embodiments, the primer concentration in the multiplex PCR is adjusted to reach high amplicon uniformity. In some embodiments, a lower concentration of primers increases the target nucleic acid ratio.

After PCR amplification, nucleic acids are quantified by counting the partitions that contain PCR amplicons. Partitioning of the sample allows quantification of the number of different molecules by assuming that the population of molecules follows a Poisson distribution. For a description of digital PCR methods, see, e.g., Hindson et al. (2011) Anal. Chem. 83(22):8604-8610; Pohl and Shih (2004) Expert Rev. Mol. Diagn. 4(1):41-47; Pekin et al. (2011) Lab Chip 11 (13): 2156-2166; Pinheiro et al. (2012) Anal. Chem. 84 (2): 1003-1011; Day et al. (2013) Methods 59(1):101-107; herein incorporated by reference in their entireties.

Oligonucleotides, including primers and probes can be readily synthesized by standard techniques, e.g., solid phase synthesis via phosphoramidite chemistry, as disclosed in U.S. Pat. Nos. 4,458,066 and 4,415,732, incorporated herein by reference; Beaucage et al. Tetrahedron (1992) 4:2223-2311; and Applied Biosystems User Bulletin No. 13 (1 Apr. 1987). Other chemical synthesis methods include, for example, the phosphotriester method described by Narang et al. Meth. Enzymol. (1979) 6:90 and the phosphodiester method disclosed by Brown et al. Meth. Enzymol. (1979) 68:109. Poly(A) or poly(C), or other non-complementary nucleotide extensions may be incorporated into oligonucleotides using these same methods. Hexaethylene oxide extensions may be coupled to the oligonucleotides by methods known in the art. Cload et al. J. Am. Chem. Soc. (1991) 113:6324-6326; U.S. Pat. No. 4,914,210 to Levenson et al.; Durand et al. Nucleic Acids Res. (1990)1:6353-6359; and Horn et al. Tet. Lett. (1986) 27:4705-4708. Moreover, the oligonucleotides (e.g., primers and probes) may be coupled to labels for detection.

There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al. Nucl. Acids Res. (1978)5:363-384 which discloses the use of ferritin-avidin-biotin labels; and Chollet et al. Nucl. Acids Res. (1985) 1:1529-1541 which discloses biotinylation of the 5 termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987)15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al. Nucl. Acids Res. (1985) 13:4485-4502 and Spoat et al. Nucl. Acids Res. (1987) 15:4837-4848. A comprehensive review of methodologies for labeling DNA fragments is provided in Matthews et al. Anal. Biochem. (1988) 169:1-25.

For example, oligonucleotides may be fluorescently labeled by linking a fluorescent molecule to the non-ligating terminus of the molecule. Guidance for selecting appropriate fluorescent labels can be found in Smith et al. Meth. Enzymol. (1987) 155:260-301; Karger et al. Nucl. Acids Res. (1991) 19:4955-4962; Guo et al. (2012) Anal. Bioanal. Chem. 402(10):3115-3125; and Molecular Probes Handbook, A Guide to Fluorescent Probes and Labeling Technologies, 11^{th} edition, Johnson and Spence eds., 2010 (Molecular Probes/Life Technologies). Fluorescent labels include fluorescein and derivatives thereof, such as disclosed in U.S. Pat. No. 4,318,846 and Lee et al. Cytometry (1989)10:151-164. Dyes for use in the present invention include 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Additional dyes include SYBR green, SYBR gold, Yakima Yellow, Texas Red, 3-(ε-carboxypentyl)-3′-ethyl-5,5′-dimethyloxa-carbocyanine (CYA); 6-carboxy fluorescein (FAM); CAL Fluor Orange 560, CAL Fluor Red 610, Quasar Blue 670; 5,6-carboxyrhodamine-110 (R110); 6-carboxyrhodamine-6G (R6G); N′,N′,N′,N′-tetramethyl-6-carboxyrhodamine (TAMRA); 6-carboxy-X-rhodamine (ROX); 2′, 4′, 5′, 7′,-tetrachloro-4-7-dichlorofluorescein (TET); 2′, 7′-dimethoxy-4′, 5′-6 carboxyrhodamine (JOE); 6-carboxy-2′,4,4′,5′,7,7′-hexachlorofluorescein (HEX); Dragonfly orange; ATTO-Tec; Bodipy; ALEXA; VIC, Cy3, and Cy5. These dyes are commercially available from various suppliers such as Life Technologies (Carlsbad, Calif.), Biosearch Technologies (Novato, Calif.), and Integrated DNA Technolgies (Coralville, Iowa). Fluorescent labels include fluorescein and derivatives thereof, such as disclosed in U.S. Pat. No. 4,318,846 and Lee et al. Cytometry (1989)100:151-164, and 6-FAM, JOE, TAMRA, ROX, HEX-1, HEX-2, ZOE, TET-1 or NAN-2, and the like.

Oligonucleotides can also be labeled with a minor groove binding (MGB) molecule, such as disclosed in U.S. Pat. Nos. 6,884,584, 5,801,155; Afonina et al. (2002) Biotechniques 32:940-944, 946-949; Lopez-Andreo et al. (2005) Anal. Biochem. 339:73-82; and Belousov et al. (2004) Hum Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson et al. (1995) “Detection of Acridinium Esters by Chemiluminescence” in Nonisotopic Probing. Blotting and Sequencing, Kricka L. J. (ed.) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al. Clin. Chem. (1983) 29:1474-1479; Berry et al. Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

### Adapters

Methods of the present invention involve attaching an adapter to a nucleic acid (e.g., a nucleic acid (e.g., a lhbrary fragment of a NGS library or an amplicon of an amplicon library). In certain embodiments, the adapters are attached to a nucleic acid with an enzyme. The enzyme may be a ligase or a polymerase. The ligase may be any enzyme capable of ligating an oligonucleotide (single stranded RNA, double stranded RNA, single stranded DNA, or double stranded DNA) to another nucleic acid molecule. Suitable ligases include T4 DNA ligase and T4 RNA ligase (such ligases are available commercially, e.g., from New England Biolabs). Methods for using ligases are well known in the art. The ligation may be blunt-ended or via use of complementary over hanging ends. In certain embodiments, the ends of nucleic acids may be phosphorylated (e.g., using T4 polynucleotide kinase), repaired, trimmed (e.g. using an exonuclease), or filled (e.g., using a polymerase and dNTPs), to form blunt ends. Upon generating blunt ends, the ends may be treated with a polymerase and dATP to form a template independent addition to the 3′ end of the fragments, thus producing a single A overhanging. This single A is used to guide ligation of fragments with a single T overhanging from the 5′ end in a method referred to as T-A cloning. The polymerase may be any enzyme capable of adding nucleotides to the 3′ and the 5′ terminus of template nucleic acid molecules.

In some embodiments, the adapters comprise a universal sequence and/or an index. e.g., a barcode nucleotide sequence. Additionally, adapters can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters (e.g., a universal sequence), one or more restriction enzyme recognition sites, one or more overhangs complementary to one or more target polynucleotide overhangs, one or more probe binding sites (e.g. for attachment to a sequencing platform, such as a flow cell for massive parallel sequencing, such as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′end, at or near the 5′ end, or in the interior of the adapter oligonucleotide. When an adapter oligonucleotide is capable of forming secondary structure, such as a hairpin, sequence elements can be located partially or completely outside the secondary structure, partially or completely inside the secondary structure, or in between sequences participating in the secondary structure. For example, when an adapter oligonucleotide comprises a hairpin structure, sequence elements can be located partially or completely inside or outside the hybridizable sequences (the “stem”), including in the sequence between the hybridizable sequences (the “loop”). In some embodiments, the first adapter oligonucleotides in a plurality of first adapter oligonucleotides having different barcode sequences comprise a sequence element common among all first adapter oligonucleotides in the plurality. In some embodiments, all second adapter oligonucleotides comprise a sequence element common among all second adapter oligonucleotides that is different from the common sequence element shared by the first adapter oligonucleotides. A difference in sequence elements can be any such that at least a portion of different adapters do not completely align, for example, due to changes in sequence length, deletion or insertion of one or more nucleotides, or a change in the nucleotide composition at one or more nucleotide positions (such as a base change or base modification).

In some embodiments, an adapter oligonucleotide comprises a 5′ overhang, a 3′ overhang, or both that is complementary to one or more target polynucleotides. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

In some embodiments, the adapter sequences can contain a molecular binding site identification element to facilitate identification and isolation of the target nucleic acid for downstream applications. Molecular binding as an affinity mechanism allows for the interaction between two molecules to result in a stable association complex. Molecules that can participate in molecular binding reactions include proteins, nucleic acids, carbohydrates, lipids, and small organic molecules such as ligands, peptides, or drugs.

When a nucleic acid molecular binding site is used as part of the adapter, it can be used to employ selective hybridization to isolate a target sequence. Selective hybridization may restrict substantial hybridization to target nucleic acids containing the adapter with the molecular binding site and capture nucleic acids that are sufficiently complementary to the molecular binding site. Thus, through “selective hybridization” one can detect the presence of the target polynucleotide in an un-pure sample containing a pool of many nucleic acids. An example of a nucleotide-nucleotide selective hybridization isolation system comprises a system with several capture nucleotides that comprise complementary sequences to the molecular binding identification elements and are optionally immobilized to a solid support. In other embodiments, the capture polynucleotides could be complementary to the target sequences itself or a barcode or unique tag contained within the adapter. The capture polynucleotides can be immobilized to various solid supports, such as inside of a well of a plate, mono-dispersed spheres, microarrays, or any other suitable support surface known in the art. The hybridized complementary adapter polynucleotides attached on the solid support can be isolated by washing away the undesirable non-binding nucleic acids, leaving the desirable target polynucleotides behind. If complementary adapter molecules are fixed to paramagnetic spheres or similar bead technology for isolation, then spheres can be mixed in a tube together with the target polynucleotide containing the adapters. When the adapter sequences have been hybridized with the complementary sequences fixed to the spheres, undesirable molecules can be washed away while spheres are kept in the tube with a magnet or similar agent. The desired target molecules can be subsequently released by increasing the temperature, changing the pH, or by using any other suitable elution method known in the art

### Barcodes

A barcode is a known nucleic acid sequence that allows some feature of a nucleic acid with which the barcode is associated to be identified. In some embodiments, the feature of the nucleic acid to be identified is the sample or source from which the nucleic acid is derived. The barcode sequence generally includes certain features that make the sequence useful in sequencing reactions. For example, the barcode sequences are designed to have minimal or no homopolymer regions, e.g., 2 or more of the same base in a row such as AA or CCC, within the barcode sequence. In some embodiments, the barcode sequences are also designed so that they are at least one edit distance away from the base addition order when performing base-by-base sequencing, ensuring that the first and last bases do not match the expected bases of the sequence.

In some embodiments, the barcode sequences are designed such that each sequence is correlated to a particular target nucleic acid, allowing the short sequence reads to be correlated back to the target nucleic acid from which they came. Methods of designing sets of barcode sequences are shown, for example, in U.S. Pat. No. 6,235,475, the contents of which are incorporated by reference herein in their entirety. In some embodiments, the barcode sequences range from about 5 nucleotides to about 15 nucleotides. In a particular embodiment, the barcode sequences range from about 4 nucleotides to about 7 nucleotides. Since the barcode sequences are sequenced along with the ladder fragment nucleic acid, in embodiments using longer sequences the barcode length is of a minimal length so as to permit the longest read from the fragment nucleic acid attached to the barcode. In some embodiments, the barcode sequences are spaced from the fragment nucleic acid molecule by at least one base. e.g., to minimize homopolymeric combinations.

In some embodiments, lengths and sequences of barcode sequences are designed to achieve a desired level of accuracy of determining the identity of nucleic acid. For example, in some embodiments barcode sequences are designed such that after a tolerable number of point mutations, the identity of the associated nucleic acid can still be deduced with a desired accuracy. In some embodiments, a Tn-5 transposase (commercially available from Epicentre Biotechnologies; Madison, Wis.) cuts a nucleic acid into fragments and inserts short pieces of DNA into the cuts. The short pieces of DNA are used to incorporate the barcode sequences.

Attaching adaptors comprising barcodes to nucleic acid templates is shown in U.S. Pat. Appl. Pub. No. 2008/0081330 and in International Pat. Appl. No. PCT/US09/64001, the content of each of which is incorporated by reference herein in its entirety. Methods for designing sets of barcode sequences and other methods for attaching adaptors (e.g., comprising barcode sequences) are shown in U.S. Pat. Nos. 6,138,077; 6,352,828; 5,636,400; 6,172,214; 6,235,475; 7,393.665; 7.544.473; 5.846.719; 5,695,934; 5,604,097; 6,150,516; RE39,793; 7,537,897; 6172.218; and 5,863,722, the content of each of which is incorporated by reference herein in its entirety. In certain embodiments, a single barcode is attached to each fragment. In other embodiments, a plurality of barcodes, e.g., two barcodes, is attached to each fragment

### Nucleic Acid Sequencing

In some embodiments of the present invention, nucleic acid sequence data are generated. Various embodiments of nucleic acid sequencing platforms (e.g., a nucleic acid sequencer) include components as described below. According to various embodiments, a sequencing instrument includes a fluidic delivery and control unit, a sample processing unit, a signal detection unit, and a data acquisition, analysis, and control unit. Various embodiments of the instrument provide for automated sequencing that is used to gather sequence Information from a plurality of sequences in parallel and/or substantially simultaneously.

In some embodiments, the fluidics delivery and control unit includes a reagent delivery system. The reagent delivery system includes a reagent reservoir for the storage of various reagents. The reagents can include RNA-based primers, forward/reverse DNA primers, nucleotide mixtures (e.g., in some embodiments, compositions comprise nucleotide analogs) for sequencing-by-synthesis, buffers, wash reagents, blocking reagents, stripping reagents, and the like. Additionally, the reagent delivery system can include a pipetting system or a continuous flow system that connects the sample processing unit with the reagent reservoir.

In some embodiments, the sample processing unit includes a sample chamber, such as flow cell, a substrate, a micro-array, a multi-well tray, or the like. The sample processing unit can include multiple lanes, multiple channels, multiple wells, or other means of processing multiple sample sets substantially simultaneously Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber. In some embodiments, the signal detection unit can include an imaging or detection sensor. For example, the imaging or detection sensor (e.g., a fluorescence detector or an electrical detector) can include a CCD, a CMOS, an ion sensor, such as an ion sensitive layer overlying a CMOS, a current detector, or the like. The signal detection unit can include an excitation system to cause a probe, such as a fluorescent dye, to emit a signal. The detection system can include an illumination source, such as an are lamp, a laser, a light emitting diode (LED), or the like. In particular embodiments, the signal detection unit includes optics for the transmission of light from an illumination source to the sample or from the sample to the imaging or detection sensor. Alternatively, the signal detection unit may not include an illumination source, such as for example, when a signal is produced spontaneously as a result of a sequencing reaction. For example, a signal can be produced by the interaction of a released moiety, such as a released ion interacting with an ion-sensitive layer, or a pyrophosphate reacting with an enzyme or other catalyst to produce a chemiluminescent signal. In another example, changes in an electrical current, voltage, or resistance are detected without the need for an illumination source.

In some embodiments, a data acquisition analysis and control unit monitors various system parameters. The system parameters can include temperatures of various portions of the instrument, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of the instruments and systems are used to practice sequencing methods such as sequencing by synthesis, single molecule methods, and other sequencing techniques. Sequencing by synthesis can include the incorporation of dye labeled nucleotides, chain termination, ion/proton sequencing, pyrophosphate sequencing, or the like. Single molecule techniques can include staggered sequencing, where the sequencing reaction is paused to determine the identity of the incorporated nucleotide.

In some embodiments, the sequencing instrument determines the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In some embodiments, the nucleic acid can include or be derived from a fragment library, an amplicon library, a mate pair library, a ChIP fragment, or the like. In particular embodiments, the sequencing instrument can obtain the sequence information from a single nucleic acid molecule or from a group of substantially identical nucleic acid molecules.

### Next-Generation Sequencing

Particular sequencing technologies contemplated by the technology are next-generation sequencing (NGS) methods that share the common feature of massively parallel, high-throughput strategies, with the goal of lower costs in comparison to older sequencing methods (see, e.g., Voelkerding et al., Clinical Chem., 55: 641-658, 2009; MacLean et al, Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. Nos. 6,210,891; 6,258,568; each herein incorporated by reference in its entirety), the NGS fragment library is clonally amplified in-situ by capturing single template molecules with beads bearing oligonucleotides complementary to the adapters. Each bead bearing a single template type is compartmentalized into a water-m-oil microvesicle, and the template is clonally amplified using a technique referred to as emulsion PCR, the emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and a luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev Microbiol., 7: 287-296; U.S. Pat. Nos. 6,833,246; 7,115,400; 6,969,488; each herein incorporated by reference in its entirety), sequencing data are produced in the form of shorter-length reads. In this method, the fragments or amplicons of the NGS library are captured on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 100 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. Nos. 5,912,148; 6,130,073; each herein incorporated by reference in their entirety) also involves clonal amplification of the NGS fragment library by emulsion PCR. Following this, beads bearing template are immobilized on a derivatized surface of a glass flow-cell, and a primer complementary to the adapter oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLD system, interrogation probes have 16 possible combinations of the two bases at the 3′ end of each probe, and one of four fluors at the 5′ end Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, HeliScope by Helicos BioSciences is employed (Voelkerding et al., Clinical Chem., 55: 641-658, 2009; MacLean et al., Nature Rev Microbiol., 7: 287-296; U.S. Pat. Nos. 7,169,560; 7,282,337; 7,482,120; 7,501,245; 6,818,395; 6,911,345; 7,501,245; each herein incorporated by reference in their entirety). HeliScope sequencing is achieved by addition of polymerase and serial addition of fluorescently-labeled dNTP reagents. Incorporation events result in a fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In some embodiments, 454 sequencing by Roche is used (Margulies et al. (2005) Nature 437: 376-380) 454 sequencing involves two steps. In the first step, DNA is sheared into fragments of approximately 300-800 base pairs and the fragments are blunt ended Oligonucleotide adapters are then ligated to the ends of the fragments. The adapters serve as primers for amplification and sequencing of the fragments. The fragments can be attached to DNA capture beads, e.g., streptavidin-coated beads using, e.g., an adapter that contains a 5′-biotin tag. The fragments attached to the beads are PCR amplified within droplets of an oil-water emulsion. The result is multiple copies of clonally amplified DNA fragments on each bead. In the second step, the beads are captured in wells (picoliter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated Pyrosequencing makes use of pyrophosphate (PPi) which is released upon nucleotide addition PPi is converted to ATP by ATP sulfurylase in the presence of adenosine 5′ phosphosulfate. Luciferase uses ATP to convert luciferin to oxylucifenn, and this reaction generates light that is detected and analyzed.

The on Torrent technology is a method of DNA sequencing based on the detection of hydrogen ions that are released during the polymerization of DNA (see, e.g., Science 327(5970): 1190 (2010); U S. Pat. Appl. Pub. Nos. 20090026082, 20090127589, 20100301398, 20100197507, 20100188073, and 20100137143, incorporated by reference in their entireties for all purposes). A microwell contains a fragment of the NGS library to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers the ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ^{˜}99.6% for 50 base reads, with ^{˜}100 Mb generated per run. The read-length is 100 base pairs. The accuracy for homopolymer repeats of 5 repeats in length is 98%. The benefits of ion semiconductor sequencing are rapid sequencing speed and low upfront and operating costs.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid Reporter elements of the Xpandomer are then detected Additional details relating to Xpandomer-based approaches are described in, for example, U.S. Pat. Pub No. 2009/0035777, entitled “High throughput nucleic acid sequencing by expansion,” filed Jun. 19, 2008, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781.166; each herein incorporated by reference in their entirety) in which fragments of the NGS library are immobilized, primed, then subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al. Clinical Chem., 55: 641-658, 2009; MacLean et al., Nature Rev. Microbiol., 7, 287-296, U.S. Pat. Nos. 7,170,050; 7,302,146; 7,313,308. U.S. Pat. No. 7,476,503; all of which are herein incorporated by reference) utilizes reaction wells 50-100 nm in diameter and encompassing a reaction volume of approximately 20 zeptoliters (10⁻²¹ liters). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, single molecule real time (SMRT) DNA sequencing methods using zero-mode waveguides (ZMWs) developed by Pacific Biosciences. or similar methods, are employed With this technology, DNA sequencing is performed on SMRT chips, each containing thousands of zero-mode waveguides (ZNWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters. At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations that promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

In some embodiments, nanopore sequencing is used (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

In some embodiments, a sequencing technique uses a chemical-sensitive field effect transistor (chemFET) array to sequence DNA (for example, as described in US Patent Application Publication No. 20090026082). In one example of the technique, DNA molecules are placed into reaction chambers, and the template molecules are hybridized to a sequencing primer bound to a polymerase. Incorporation of one or more triphosphates into a new nucleic acid strand at the 3′end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, sequencing technique uses an electron microscope (Moudrianakis E. N. and Beer M. Proc Natl Acad Sci USA. 1965 March; 53.564-71). In one example of the technique, individual DNA molecules are labeled using metallic labels that are distinguishable using an electron microscope. These molecules are then stretched on a flat surface and imaged using an electron microscope to measure sequences.

In some embodiments, “four-color sequencing by synthesis using cleavable fluorescent nucleotide reversible terminator” as described in Turro, et al. PNAS 103-19635-40 (20)6) is used, e.g., as commercialized by Intelligent Bio-Systems. The technology described in U.S. Pat. Appl. Pub. Nos. 2010/0323350, 2010/0063743, 2010/0159531, 20100035253, 20100152050, incorporated herein by reference for all purposes.

In some embodiments, the quality of data produced by a next-generation sequencing platform depends on the concentration of DNA (e.g. an NGS library such as a fragment library or an amplicon panel library) that is loaded onto the sequencer work flow clonal amplification step. For instance, loading a concentration that is below a minimal threshold may result in low or sub-optimal sequencer output while loading a concentration that is above a maximum threshold may result in low quality sequence or no sequencer output. Accordingly, the present invention provided herein finds use in preparing a sample having an appropriate concentration for sequencing, e.g., such that the sequence data that is output has a desirable quality.

Any high-throughput technique for sequencing the nucleic acids can be used in the practice of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina MiSeq, NextSeq, and HiSeq platforms, which use reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference).

### Nucleic Acid Sequence Analysis

In some embodiments, a computer-based analysis program is used to translate the raw data generated by the detection assay (e.g., sequencing reads) into data of predictive value for an end user (e.g., medical personnel). The user can access the predictive data using any suitable means. Thus, in some preferred embodiments, the present invention provides the further benefit that the user, who is not likely to be trained in genetics or molecular biology, need not understand the raw data. The data is presented directly to the end user in its most useful form. The user is then able to immediately utilize the information to determine useful information (e.g., in medical diagnostics, research, or screening).

Some embodiments provide a system for reconstructing a nucleic acid sequence. The system can include a nucleic acid sequencer, a sample sequence data storage, a reference sequence data storage, and an analytics computing device/server/node. In some embodiments, the analytics computing device/server/node can be a workstation, mainframe computer, personal computer, mobile device, etc. The nucleic acid sequencer can be configured to analyze (e.g., interrogate) a nucleic acid fragment (e.g., single fragment, mate-pair fragment, paired-end fragment, etc.) utilizing all available varieties of techniques, platforms or technologies to obtain nucleic acid sequence information, in particular the methods as described herein using compositions provided herein. In some embodiments, the nucleic acid sequencer is in communications with the sample sequence data storage either directly via a data cable (e.g., serial cable, direct cable connection, etc.) or bus linkage or, alternatively, through a network connection (e.g., Internet, LAN. WAN, VPN, etc.). In some embodiments, the network connection can be a “hardwired” physical connection. For example, the nucleic acid sequencer can be communicatively connected (via Category 5 (CAT5), fiber optic or equivalent cabling) to a data server that is communicatively connected (via CAT5, fiber optic, or equivalent cabling) through the Internet and to the sample sequence data storage. In some embodiments, the network connection is a wireless network connection (e.g., Wi-Fi, WLAN, etc.), for example, utilizing an 802.11 a/b/g/n or equivalent transmission format. In practice, the network connection utilized is dependent upon the particular requirements of the system. In some embodiments, the sample sequence data storage is an integrated part of the nucleic acid sequencer.

In some embodiments, the sample sequence data storage is any database storage device, system, or implementation (e.g., data storage partition, etc.) that is configured to organize and store nucleic acid sequence read data generated by nucleic acid sequencer such that the data can be searched and retrieved manually (e.g., by a database administrator or client operator) or automatically by way of a computer program, application, or software script. In some embodiments, the reference data storage can be any database device, storage system, or implementation (e.g., data storage partition, etc.) that is configured to organize and store reference sequences (e.g., whole or partial genome, whole or partial exome. SNP, gen, etc.) such that the data can be searched and retrieved manually (e.g., by a database administrator or client operator) or automatically by way of a computer program, application, and/or software script. In some embodiments, the sample nucleic acid sequencing read data can be stored on the sample sequence data storage and/or the reference data storage in a variety of different data file types/formats, including, but not limited to: *.txt, *.fasta, *.csfasta, *seq.txt, *qseq.txt, * fastq, *.sff, *prb.txt, *.sms, *srs and/or * .qv.

In some embodiments, the sample sequence data storage and the reference data storage are independent standalone devices/systems or implemented on different devices. In some embodiments, the sample sequence data storage and the reference data storage are implemented on the same device/system. In some embodiments, the sample sequence data storage and/or the reference data storage can be implemented on the analytics computing device/server/node. The analytics computing device/server/node can be in communications with the sample sequence data storage and the reference data storage either directly via a data cable (e.g., serial cable, direct cable connection, etc.) or bus linkage or, alternatively, through a network connection (e.g., Internet, LAN, WAN, VPN, etc.). In some embodiments, analytics computing device/server/node can host a reference mapping engine, a de novo mapping module, and/or a tertiary analysis engine. In some embodiments, the reference mapping engine can be configured to obtain sample nucleic acid sequence reads from the sample data storage and map them against one or more reference sequences obtained from the reference data storage to assemble the reads into a sequence that is similar but not necessarily identical to the reference sequence using all varieties of reference mapping/alignment techniques and methods. The reassembled sequence can then be further analyzed by one or more optional tertiary analysis engines to identify differences in the genetic makeup (genotype), gene expression or epigenetic status of individuals that can result in large differences in physical characteristics (phenotype). For example, in some embodiments, the tertiary analysis engine can be configured to identify various genomic variants (in the assembled sequence) due to mutations, recombination/crossover or genetic drift. Examples of types of genomic variants include, but are not limited to: single nucleotide polymorphisms (SNPs), copy number variations (CNVs), insertions/deletions (Indels), inversions, etc. The optional de novo mapping module can be configured to assemble sample nucleic acid sequence reads from the sample data storage into new and previously unknown sequences. It should be understood, however, that the various engines and modules hosted on the analytics computing device/server/node can be combined or collapsed into a single engine or module, depending on the requirements of the particular application or system architecture. Moreover, m some embodiments, the analytics computing device/server/node can host additional engines or modules as needed by the particular application or system architecture.

In some embodiments, the mapping and/or tertiary analysis engines are configured to process the nucleic acid and/or reference sequence reads in color space. In some embodiments, the mapping and/or tertiary analysis engines are configured to process the nucleic acid and/or reference sequence reads in base space. It should be understood, however, that the mapping and/or tertiary analysis engines disclosed herein can process or analyze nucleic acid sequence data in any schema or format as long as the schema or format can convey the base identity and position of the nucleic acid sequence.

Furthermore, a client terminal can be a thin client or thick client computing device. In some embodiments, client terminal can have a web browser that can be used to control the operation of the reference mapping engine, the de novo mapping module and/or the tertiary analysis engine. That is, the client terminal can access the reference mapping engine, the de novo mapping module and/or the tertiary analysis engine using a browser to control their function. For example, the client terminal can be used to configure the operating parameters (e.g., mismatch constraint, quality value thresholds, etc.) of the various engines, depending on the requirements of the particular application. Similarly, client terminal can also display the results of the analysis performed by the reference mapping engine the de novo mapping module and/or the tertiary analysis engine.

The present invention also encompasses any method capable of receiving, processing, and transmitting the information to and from laboratories conducting the assays, information provides, medical personal, and subjects.

### Applications/Uses

The present invention is not limited to particular uses, but finds use in a wide range of research (basic and applied), clinical, medical, and other biological, biochemical, and molecular biological applications. The methods and compositions of the present invention finds use in methods, kits, systems, etc. that are associated with providing a sample of nucleic acid that is concentration normalized. Some exemplary uses of the methods and compositions of the present invention include genetics, genomics, and/or genotyping. e.g., of plants, animals, and other organisms, e.g., to identify haplotypes, phasing, and/or linkage of mutations and/or alleles. In some embodiments, the methods of the present invention find use in sequencing related to cancer diagnosis, treatment, and therapy.

In some embodiments, the methods and compositions of the present invention may be used in the field of prenatal diagnosis, e.g., in identifying chromosomal abnormalities such as fetal aneuploidy. Other particular and non-limiting illustrative examples in the area of prenatal diagnosis include single gene disorders or genetic variations and conditions.

Genetic variations can range from a single base pair variation to a chromosomal variation, or any other variation known in the art. Genetic variations can be simple sequence repeats, short tandem repeats, single nucleotide polymorphisms, translocations, inversions, deletions, duplications, or any other copy number variations. In some embodiments, the chromosomal variation is a chromosomal abnormality. For example, the chromosomal variation can be aneuploidy, inversion, translocation, a deletion, or a duplication. A genetic variation can also be mosaic. For example, the genetic variation can be associated with genetic conditions or risk factors for genetic conditions (e.g., cystic fibrosis. Tay-Sachs disease, Huntington disease. Alzheimer disease, and various cancers). Genetic variations can also include any mutation, chromosomal abnormality, or other variation disclosed in the priority documents (e.g., aneuploidy, microdeletions, or microduplications) cited above. Genetic variations can have positive, negative, or neutral effects on phenotype. For example, chromosomal variations can include advantageous, deleterious, or neutral variations. In some embodiments, the genetic variation is a risk factor for a disease or disorder. In some embodiments, the genetic variation encodes a desired phenotypic trait.

In addition, the methods of the present invention find use in the field of infectious disease. e.g., in identifying infectious agents such as viruses, bacteria, fungi, etc., and in determining viral types, families, species, and/or quasi-species, and to identify haplotypes, phasing, and/or linkage of mutations and/or alleles. Other particular and non-limiting illustrative examples in the area of infectious disease include characterizing antibiotic resistance determinants, tracking infectious organisms for epidemiology; monitoring the emergence and evolution of resistance mechanisms; identifying species, sub-species, strains, extra-chromosomal elements, types, etc. associated with virulence, monitoring the progress of treatments, etc.

In some embodiments, the methods of the present invention find use in transplant medicine, e.g., for typing of the major histocompatibility complex (MHC), typing of the human leukocyte antigen (HLA), and for identifying haplotypes, phasing, and/or linkage of mutations and/or alleles associated with transplant medicine (e.g., to identify compatible donors for a particular host needing a transplant, to predict the chance of rejection, to monitor rejection, to archive transplant material, for medical informatics databases, etc.).

In some embodiments, the methods and compositions of the present invention find use in oncology and fields related to oncology. Particular and non-limiting illustrative examples in the area of oncology are detecting genetic and/or genomic aberrations related to cancer, predisposition to cancer, and/or treatment of cancer. For example, in some embodiments the methods and compositions of the present invention find use in detecting the presence of a mutation, polymorphism, allele, or a chromosomal translocation associated with cancer. In some embodiments, the methods and compositions of the present invention find use in cancer screening, cancer diagnosis, cancer prognosis, measuring minimal residual disease, and selecting and/or monitoring a course of treatment for a cancer.

The methods of the invention will be especially useful in genetic screening for aneuploidy and/or copy number variation associated with various diseases, structural abnormalities, and/or genetic lethality. Correction of amplification bias in sequencing data, as described herein, makes possible more accurate detection of even minor copy number variation. In particular, the methods will find use in non-invasive prenatal testing to detect fetal chromosomal aneuploidy or copy number variation. A biological sample can be collected from the mother or potential mother of an offspring prior to conception or after conception and analyzed. Detection of aneuploidy or copy number variation, as described herein, may indicate an increased risk of the offspring developing abnormally or having a disease (e.g., Down Syndrome (Trisomy 21), Edwards Syndrome (Trisomy 18), or Patau Syndrome (Trisomy 13)). The offspring may be, for example, a neonate or a fetus. In particular, this method can be used to evaluate a mother or potential mother potentially at high risk of having a child with a disease associated with aneuploidy or copy number variation, such as a mother or potential mother who has had a previous child with such a disease or a familial history of the disease, or a history of miscarriages.

The methods of the invention will also find use in genetic testing of cancerous cells. Aneuploidy and copy number variation are commonly associated with many types of cancer. Hence, genetic testing of cancerous cells or abnormal potentially precancerous cells may be useful for diagnosing a patient with a particular type of cancer or precancerous condition and determining an appropriate treatment regimen.

For genetic testing, a biological sample containing nucleic acids is collected from an individual. The biological sample is typically blood, saliva, or cells from buccal swabbing or a biopsy, but can be any sample from bodily fluids, tissue, or cells that contains genomic DNA or RNA of the individual. For prenatal testing of a fetus, the biological sample can be, for example, amniotic fluid (e.g., amniocentesis), placental tissue (e.g., chorionic villus sampling), or fetal blood (e.g., umbilical cord blood sampling). In particular, non-invasive cell-free fetal DNA in maternal blood or nucleic acids extracted from fetal cells in maternal blood (FCMB) can be used in genetic screening. The methods of the invention are also applicable to genetic screening of embryos produced by in vitro fertilization (IVF). For example, preimplantation genetic diagnosis (PGD) can be performed using the methods described herein to correct amplification bias in order to improve detection of aneuploidy and/or copy number variation in embryos prior to transfer to a mother. In certain embodiments, nucleic acids from the biological sample are isolated and/or purified prior to amplification, sequencing, and analysis using methods well-known in the art. See, e.g., Green and Sambrook Molecular Cloning: A Laboratory Manual (Cold Spring Harbor Laboratory Press; 4^{th} edition, 2012); and Current Protocols in Molecular Biology (Ausubel ed., John Wiley & Sons, 1995); herein incorporated by reference in their entireties.

Copy number variation can be evaluated based on “relative copy number” so that apparent differences in gene copy numbers in different samples are not distorted by differences in sample amounts. The relative copy number of a gene (per genome) can be expressed as the ratio of the copy number of a target gene to the copy number of a reference polynucleotide sequence in a DNA sample. The reference polynucleotide sequence can be a sequence having a known genomic copy number. Typically, the reference sequence will have a single genomic copy and is a sequence that is not likely to be amplified or deleted in the genome. It is not necessary to empirically determine the copy number of a reference sequence. Rather, the copy number may be assumed based on the normal copy number in the organism of interest. Accordingly, the relative copy number of the target nucleotide sequence in a DNA sample is calculated from the ratio of the two genes, wherein detection of copy number variation, that is, the presence of a greater or fewer number of a gene (i.e., abnormal copy number) in the subject compared to a control subject (e.g., normal, healthy subject) is diagnostic of a disease.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1: Preparation of Next-Generation Sequencing Library Using Multiplex PCR

Here we describe methods for the preparation of next-generation sequencing libraries using multiplex PCR and their application to non-invasive prenatal testing using maternal cell-free DNA to aid detection of fetal chromosomal aneuploidy.

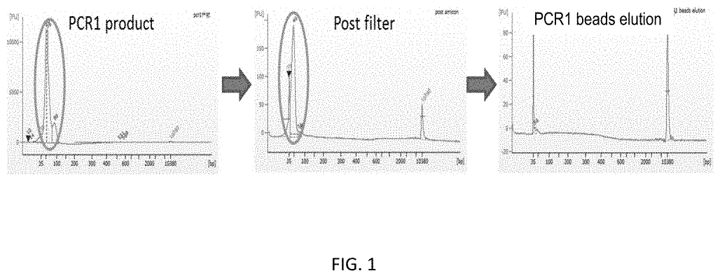

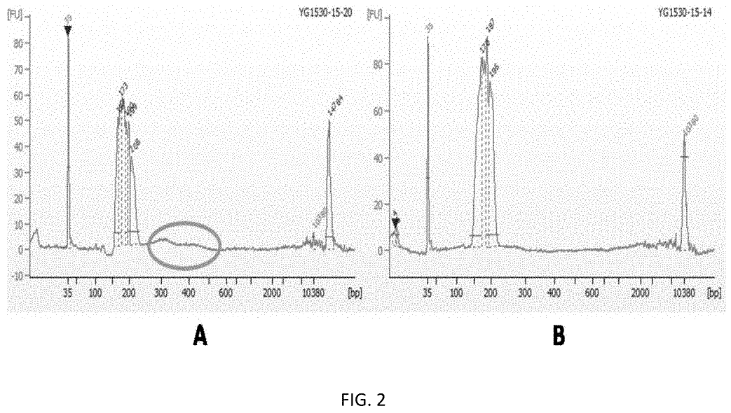

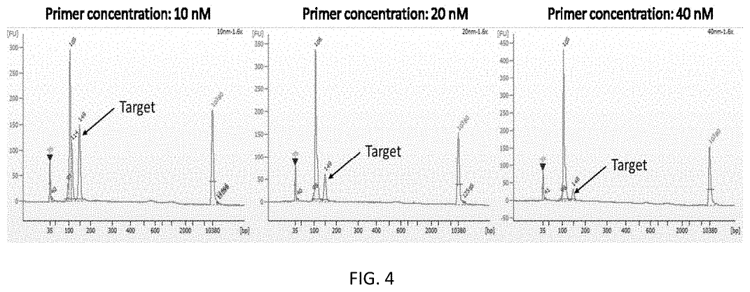

Next-generation sequencing libraries were generated as follows:
1. Nucleic acid samples were prepared as follows: plasma was isolated from maternal blood following centrifugation and cell-free DNA was obtained from the resulting plasma using a commercial DNA extraction kit.
2. the nucleic acid samples were enriched for short fragment DNA (less than 300 bp) using magnetic beads. A specific volume ratio of magnetic beads was added to the nucleic acid samples prepared in step 1 to bind 300 bp or larger DNA, the supernatant containing short DNA was removed and another specific volume ratio of magnetic beads was incubated with the supernatant to bind 200 bp or smaller DNA, the beads were washed and the short DNA was eluted from the beads for use in multiplex PCR.
3. A first multiplex PCR (more than 1,000-plex) was carried out on the enriched nucleic acid sample from step 2. PCR primer concentrations were varied to determine the effects on amplicon uniformity and target fragment ratio, the results of various primer concentrations on the amplification of nucleic acids are shown in FIG. 4.
4. the PCR amplicons from the step 3 were applied to a specific filter to eliminate unconsumed primer and primer dimers. The filtered PCR products were collected and then magnetic beads were used to selectively enrich for target amplicons based on size, the results of the enrichment are shown in FIG. 1.
5. Adapter and barcode sequences were attached to the enrich amplicons of step 4 using a second multiplex PCR. In this second PCR, the number of PCR cycles was reduced from 20 to 14 to prevent over-amplification of PCR products. FIG. 2A shows the results of 20 cycles of PCR and the over-amplification of PCR products resulting in “daisy-chain” formation. FIG. 2B shows the results of reducing PCR cycles to 14 with an improvement in the quantification of library amplicons.
6. Magnetic beads were added to the PCR amplicons from step 5 to capture target amplicons based on size. An elution buffer was mixed with the beads to elute target amplicons from the beads to generate a sequencing library for next-generation sequencing.
7. The resulting amplicon library from step 6 was subjected to next-generation sequencing.
8. The sequencing data was analyzed to determine the presence or absences of fetal chromosomal aneuploidy.

These results showed that methods and compositions of the present invention are useful for generating next-generation sequencing libraries.

### Example 2: Effects of Potassium Phosphate Concentration on Multiplex PCR

The effects of potassium phosphate concentration on multiplex PCR was determined as follows. Nucleic acid samples were prepared and subjected to multiplex PCR as described above in Example 1, except that varying concentrations of potassium phosphate (5 mM, 10 mM, and 15 mM) were used in the multiplex PCR reactions.

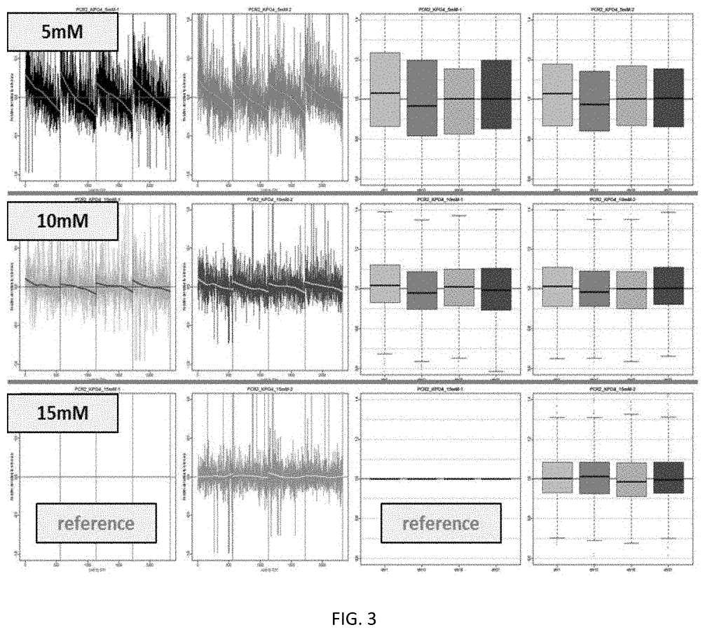

As shown in FIG. 3, potassium phosphate concentration introduced significant amplicon coverage differences between samples. Tilted fit curves shown in FIG. 3 also suggest that different potassium phosphate concentrations effect target DNA amplification.

The results showed that methods and compositions of the present invention are useful for improving amplicon coverage in multiplex PCR. These results further showed that methods and compositions of the present invention are useful for generating next-generation sequencing libraries.

### Example 3: Effects of Primer Concentration on Multiplex PCR

The effects of primer concentration on multiplex PCR was determined as follows. Nucleic acid samples were prepared and subjected to multiplex PCR as described above in Example 1, except that varying primer concentrations (10 nM, 20 nM, 40 nM) for target nucleic acids were used in the multiplex PCR reactions.

As shown in FIG. 4, a moderate lower primer concentration increased target nucleic acid amplification ratio. Lower primer concentrations also improved amplicon uniformity (see FIG. 4).

The results showed that methods and compositions of the present invention are useful for improving amplicon uniformity and target nucleic acid amplification in multiplex PCR. These results further showed that methods and compositions of the present invention are useful for generating next-generation sequencing libraries.

### Example 4: Fetal DNA Enrichment

Fetal DNA enrichment was performed as follows. Maternal blood was obtained from pregnant women and nucleic acid samples were prepared as described above in Example 1. The nucleic acid samples were enriched for short fragment DNA (less than 300 bp) using magnetic beads. A specific volume ratio of magnetic beads was added to the nucleic acid samples prepared in step 1 to bind 300 bp or larger DNA. The supernatant containing short DNA was removed and another specific volume ratio of magnetic beads was incubated with the supernatant to bind 200 bp or smaller DNA. The beads were washed and the short DNA was eluted from the beads. Fetal fraction was determined by sequencing the eluted short DNA. Fetal fraction was also determined by sequencing control maternal plasma cell-free DNA that was not subjected to the enrichment steps described above.

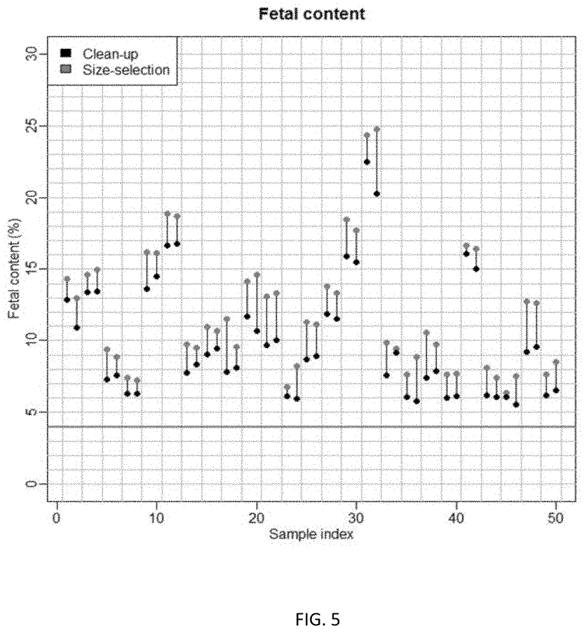

As shown in FIG. 5, size selection with magnetic beads increased fetal fraction in nucleic acid samples. These results showed that methods and compositions of the present invention are useful for enriching fetal DNA in nucleic acid samples obtained from maternal blood samples. The results suggested that the methods and compositions of the present invention would be useful for generating next-generation sequencing libraries.

### Example 5: Effects of DNA Polymerase Enzyme on Primer Dimer Formation in Multiplex PCR

The effects of DNA polymerase enzyme on primer dimer formation in multiplex PCR was determined as follows. Nucleic acid samples were prepared and subjected to multiplex PCR as described above in Example 1, except that varying DNA polymerases were used in the multiplex PCR reactions.

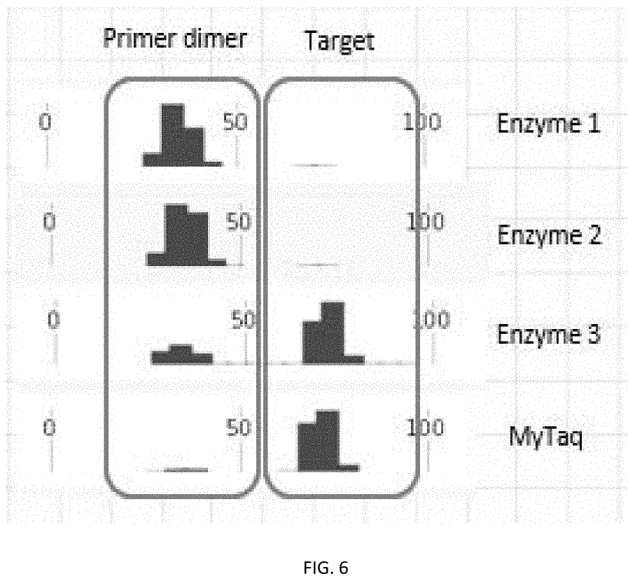

As shown in FIG. 6, the MyTaq DNA polymerase from Bioline showed the lowest amount of primer dimer formation in multiplex PCR.

These results showed that the methods and compositions of the present invention are useful for reducing primer dimer formation in multiplex PCR. These results further showed that methods and compositions of the present invention are useful for generating next-generation sequencing libraries.

Various modifications of the invention, in addition to those shown and described herein, will become apparent to those skilled in the art from the foregoing description. Such modifications are intended to fall within the scope of the appended claims.

### Example 6: Nucleic Acid Enrichment Reduces Primer Dimer Formation in Multiplex PCR

Studies to determine the effect of nucleic acid enrichment on primer dimer formation during multiplex PCR were performed as follows. Maternal blood was obtained from pregnant women and nucleic acid samples were prepared as described above in Example 1. Nucleic acid samples were enriched using 1) magnetic beads only or 2) PCR product filters and magnetic beads in series. Enriched nucleic acid samples were subjected to multiplex PCR and the amplicons were sized and quantified using a bioanalyzer.

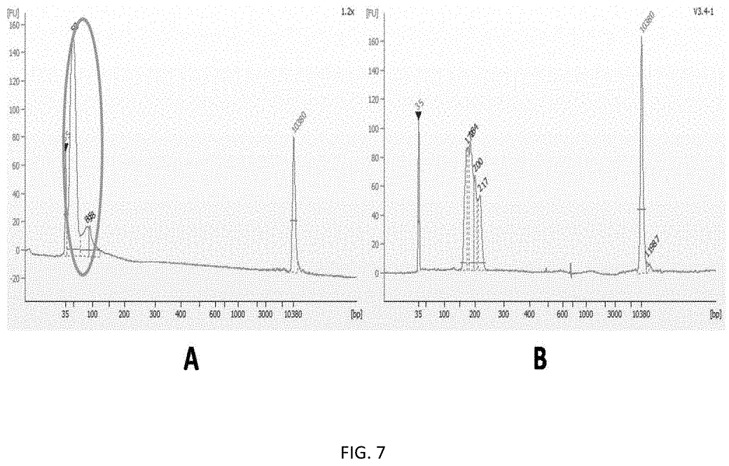

FIG. 7A shows bioanalyzer data for nucleic acid samples that were enriched using magnetic beads alone. FIG. 7B shows bioanalyzer data for nucleic acid samples that were enriched using a PCR product filter and magnetic beads in series. Enrichment with PCR product filters and magnetic beads in series reduced primer dimer formation during multiplex PCR (see FIGS. 7A-B). These results showed that methods and compositions of the present invention are useful for enriching nucleic acid samples and reducing primer dimer formation in multiplex PCR. The results suggested that the methods and compositions of the present invention would be useful for generating next-generation sequencing libraries.

All references cited herein are hereby incorporated by reference herein in their entirety.

1. A method of generating a next-generation sequencing library, the method comprising: a) providing a sample comprising nucleic acids, wherein at least some of said nucleic acids in said sample comprise target nucleic acid sequences; b) enriching said sample from step a) for said target nucleic acid sequences; c) performing a first multiplex PCR comprising target nucleic acid sequences to provide amplicons; d) enriching said sample from step c) for target amplicons; and e) performing a second multiplex PCR comprising said target amplicons, sequencing adaptors, and barcodes to form barcoded target amplicons, thereby generating a next-generation sequencing library.

2. A method of generating a next-generation sequencing library, the method comprising: a) providing a sample comprising nucleic acids, wherein at least some of said nucleic acids in said sample comprise target nucleic acid sequences; b) enriching said sample from step a) for said target nucleic acid sequences; c) performing a first multiplex PCR comprising target nucleic acid sequences to provide amplicons; d) enriching said sample from step c) for target amplicons; e) performing a second multiplex PCR comprising said target amplicons, sequencing adaptors, and barcodes to form barcoded target amplicons; and f) enriching said barcoded target amplicons from step e), thereby generating a next-generation sequencing library.

3. The method of claim 1, wherein said target nucleic acid sequences comprise 1 to 300 nucleotides.

4. The method of claim 1, wherein said enriching step comprises contacting the sample with magnetic beads, wherein said beads bind to target nucleic acid sequences in the sample; and separating the target nucleic acid sequences bound to said beads from the remaining sample.

5. The method of claim 1, wherein said first or second multiplex PCR comprises more than one primer pair and a hot-start polymerase.

6. The method of claim 5, wherein said primer pair comprises a universal sequence and a target sequence.

7. The method of claim 1, wherein said amplicons comprise a universal sequence and a target sequence.

8. The method of claim 1, wherein said enriching step comprises applying amplicons to a filter, wherein the filter substantially retains the amplicons but allows unconsumed primers and primer dimers to pass through the filter.

9. The method of claim 8, wherein the filter is a PCR products filter.

10. The method of claim 1, wherein said enriching step comprises applying amplicons, primer dimers and/or unconsumed primers to a filter to provide filtered amplicons, primer dimers and/or unconsumed primers and contacting said filtered amplicons, primer dimers and/or unconsumed primers with magnetic beads, wherein said beads bind to said filtered amplicons; and separating the filtered amplicons bound to said beads from primer dimers and/or unconsumed primers not bound to said beads.

11. The method of claim 1, wherein said second multiplex PCR comprises forward primers and reverse primers.

12. The method of claim 11, wherein the reverse primers comprise a sequencing adaptor and a universal sequence.

13. The method of claim 11, wherein the reverse primers comprise a sequencing adaptor, a barcode sequence, and a universal sequence.

14. The method of claim 11, wherein forward primers comprise a sequencing adaptor and a universal sequence.

15. The method of claim 11, wherein the forward primers comprise a sequencing adaptor, a barcode sequence, and a universal sequence.

16. The method of claim 1, wherein enriching said barcoded target amplicons comprises contacting the barcoded target amplicons, primer dimers and/or unconsumed primers with magnetic beads, wherein said beads bind to said barcoded target amplicons; and separating the barcoded target amplicons bound to said beads from primer dimers and unconsumed primers not bound to said beads.

17. The method of claim 1, wherein said enriching step comprises contacting the nucleic acids and target nucleic acids with magnetic beads, wherein said beads bind to said nucleic acids but do not bind to said target nucleic acids; and separating the nucleic acids bound to said beads from said target nucleic acids not bound to said beads.

18. The method of claim 1, wherein said enriching step comprises contacting the target nucleic acids, primer dimers, dNTPs, and/or primers with a filter, wherein said filter retains target nucleic acids but not primer dimers, dNTPs, and/or primers.

19. The method of claim 18, wherein the filter is a PCR products filter.

20. The method of claim 1, wherein said enriching step comprises subjecting the target nucleic acids to gel electrophoresis, ethanol precipitation, or column chromatography.

21-34. (canceled)
